(19) United States
(12) Patent Application Publication
(10) Pub. No.: US 2025/0314579 A1
(43) Pub. Date: Oct. 9, 2025

(54) CORROSION ESTIMATION DEVICE AND METHOD

(71) Applicant: Nippon Telegraph and Telephone Corporation, Tokyo (JP)

(72) Inventors: Shota Oki, Tokyo (JP); Mamoru Mizunuma, Tokyo (JP); Shingo Mineta, Tokyo (JP); Masayuki Tsuda, Tokyo (JP)

(21) Appl. No.: 18/865,790

(22) PCT Filed: May 17, 2022
(86) PCT No.: PCT/JP2022/020487

§ 371 Date: Nov. 14, 2024

(51) Int. Cl. G01N 17/02; G01N 33/24

(52) U.S. Cl. CPC G01N 17/02; G01N 33/246

(57) ABSTRACT

In a first step, a pretreatment for measuring a moisture characteristic of soil is performed. The pretreatment can be removal of moisture from the soil of interest. For example, removing water that is a cause of a soil mass can be the pretreatment. Next, in a second step, the moisture characteristic of the soil subjected to the pretreatment is measured. The moisture characteristic is a water content of the soil. In addition, the moisture characteristic is a matrix potential value at a specified soil water content. Next, in a third step, the corrosion of the metal member buried in the soil is estimated from the measured moisture characteristic.

### CROSS-REFERENCE TO RELATED APPLICATIONS

This application is a national phase entry of PCT Application No. PCT/JP2022/020487, filed on May 17, 2022, which application is hereby incorporated herein by reference.

### TECHNICAL FIELD

The present invention relates to a corrosion estimation device and a corrosion estimation method for estimating corrosion of a metal member buried in soil.

### BACKGROUND

Social infrastructure facilities that support our life have been rapidly developed since the high economic growth period. For this reason, it is said that in 2030, facilities that are 50 years old after construction will occupy more than half of the entire facilities. To prevent failure of these aging infrastructure facilities, maintenance operation by periodic inspection has been conventionally performed. However, in recent years, inspection work has been delayed due to an increase in aging facilities and a decrease in inspection technicians, and appropriate measures cannot be taken for objects subject to deterioration to be inspected, which may cause a serious accident such as collapse. Further, since visual inspection is difficult depending on an installation place of the facility, the inspection itself is not performed in many cases. A typical example of the place where the visual inspection is difficult is in soil.

Therefore, in recent years, research has been actively conducted for establishing a technique of predicting and estimating a deterioration state of a facility buried in soil. If this prediction and estimation technique is established, it becomes possible to distinguish between a subject with severe deterioration and a subject with slow deterioration without performing a site inspection, and not only safety is secured by preferentially updating a subject with fast deterioration progression, but also efficiency in terms of cost is expected to be improved by using a subject with slow deterioration progression for a longer time. Since the main cause of deterioration of a buried steel material is soil corrosion, it is important to grasp the relationship between a dominant factor of soil corrosion and a corrosion rate in order to predict and estimate the deterioration state.

However, soil is a special environment in which three phases of a solid phase, a gas phase, and a liquid phase coexist, and it is considered that there are various factors contributing to a corrosion reaction (Non Patent Literature 1). In particular, solid phase information specific to soil can be important information for understanding soil corrosion. Examples of this unique information include water that determines a presence or absence of occurrence of the corrosion reaction, and a soil particle size distribution as solid phase information that affects a state of oxygen.

A soil particle gap structure and a particle packing density change depending on a difference in the particle size distribution, which greatly affects ease of oxygen supply from a soil surface layer and a wetted area of a surface of the metal member by water captured by capillary action. In addition, a pressure of the water captured by capillary action is also different due to a smallness of the soil particle gap, and under a condition that the water is captured with a stronger pressure, a thin water film is formed on the surface of the metal member, and an environment in which soil corrosion easily proceeds is generated. Therefore, it is important to utilize the particle size distribution of soil in order to predict and estimate soil corrosion.

Regarding the particle size distribution, useful information can be read from a frequency distribution curve represented by a horizontal axis particle size and a vertical axis frequency. For example, the size of the particle size distribution can be evaluated by a shape of the curve, and in a case where the shape of the frequency distribution curve is sharp, it means that a large amount of soil having the same particle size is included because the frequency of a certain particle size is high, and it can be seen that the distribution of the particle size is narrow. Conversely, when the shape of the frequency distribution curve is broad, it can be seen that soil of any particle size is included, and the distribution of the particle size is wide. A porosity of the particles varies depending on the size of the particle size distribution, and thus not only affects a rate of drainage, but also affects the formation of the thin water film because a capturing power of water by capillary force based on a soil particle gap size varies.

### CITATION LIST

### Non Patent Literature

Non Patent Literature 1: Y. Wan et al., “Corrosion Behaviors of Q235 Steel in Indoor Soil”, International Journal of ELECTROCHEMICAL SCIENCE, vol. 8, pp. 12531-12542, 2013.

### SUMMARY

### Technical Problem

As described above, useful information can be obtained from a particle size distribution, but it is troublesome to convert a result of actually measuring the particle size distribution into a soil particle packing density and a capturing power of water by capillary force. Even if the particle size distribution can be grasped, it cannot be said that the soil particle packing and a wettability of a surface of a metal member in an actual burying environment can be accurately evaluated. As described above, conventionally, there has been a problem that accurate estimation of corrosion of a metal member buried in the ground is not easy.

Embodiments of the present invention has been made to solve the above problems, and an object thereof is to enable more accurate and easier estimation of the corrosion of the metal member buried in the ground.

### Solution to Problem

A corrosion estimation device according to embodiments of the present invention includes a soil conditioner that performs a pretreatment for measuring a moisture characteristic of soil, a measurement instrument that measures the moisture characteristic of the soil pretreated by the soil conditioner, and a calculator that estimates corrosion of a metal member buried in the soil from the moisture characteristic measured by the measurement instrument.

In addition, a corrosion estimation method according to embodiments of the present invention includes a first step of performing the pretreatment for measuring the moisture characteristic of the soil, a second step of measuring the moisture characteristic of the soil subjected to the pretreatment, and a third step of estimating the corrosion of the metal member buried in the soil from the measured moisture characteristic.

### Advantageous Effects

As described above, according to embodiments of the present invention, since the corrosion of the metal member is estimated from the moisture characteristic of the soil in which the metal member is buried, the corrosion of a metal member buried in the ground can be more accurately and easily estimated.

### BRIEF DESCRIPTION OF THE DRAWINGS

FIG. 1 is a flowchart for describing a corrosion estimation method according to an embodiment of the present invention.

FIG. 2 is a configuration diagram illustrating a configuration of a corrosion estimation device according to the embodiment of the present invention.

FIG. 3A is a configuration diagram illustrating a configuration of a soil conditioner 101.

FIG. 3B is a configuration diagram illustrating a configuration of a measurement instrument 102.

FIG. 4 is a characteristic diagram illustrating an example of a temporal change of a soil water content in a soil accommodating unit 121.

FIG. 5 is a characteristic diagram illustrating an example of a soil moisture characteristic curve representing a relationship between a matrix potential and the soil water content.

FIG. 6 is a characteristic diagram illustrating an example of a relationship between the matrix potential and a maximum corrosion rate derived by a calculator 103.

FIG. 7 is a characteristic diagram illustrating an example of a graph representing a temporal change of a corrosion rate output by the calculator 103.

FIG. 8A is a flowchart illustrating an operation example of the corrosion estimation device according to the embodiment of the present invention.

FIG. 8B is a flowchart illustrating an operation example of the corrosion estimation device according to the embodiment of the present invention.

### DETAILED DESCRIPTION OF ILLUSTRATIVE EMBODIMENTS

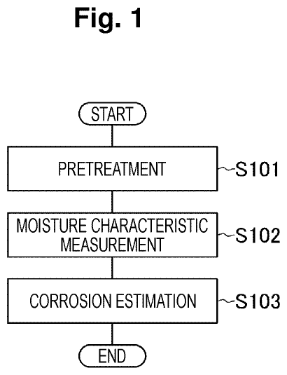

Hereinafter, a corrosion estimation method according to an embodiment of the present invention will be described with reference to FIG. 1. In this method, first, in a first step S101, a pretreatment for measuring a moisture characteristic of soil is performed. The pretreatment can be removal of moisture from the soil of interest. For example, in a case where soil collected from a place where a metal member (for example, a steel material) to be estimated for corrosion is buried is wet soil, a soil particle mass may be formed by water trapped in a soil particle gap. When the moisture characteristic is measured in the presence of the soil particle mass, an original moisture characteristic may not be evaluated because a particle size distribution is different. In such a case, as the pretreatment, it is important to remove the water that is a cause of the soil mass.

Next, in a second step S102, the moisture characteristic of the soil subjected to the pretreatment is measured. The moisture characteristic is a water content of the soil. In addition, the moisture characteristic is a matrix potential value at a specified soil water content.

Next, in a third step S103, the corrosion of the metal member buried in the soil is estimated from the measured moisture characteristic.

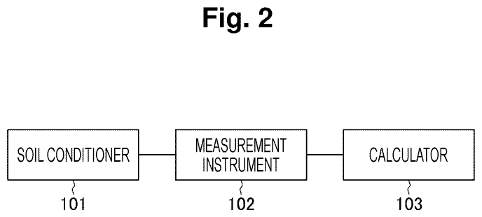

Next, a corrosion estimation device for performing the above-described corrosion estimation method will be described with reference to FIG. 2. The corrosion estimation device includes a soil conditioner 101 that performs the pretreatment for measuring the moisture characteristic of the soil, a measurement instrument 102 that measures the moisture characteristic of the soil pretreated by the soil conditioner 101, and a calculator 103 that estimates the corrosion of the metal member buried in the soil from the moisture characteristic measured by the measurement instrument 102. The measurement instrument 102 measures the water content of the soil. In addition, the measurement instrument 102 measures a matrix potential at the specified soil water content.

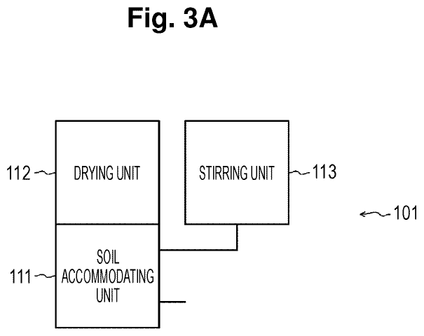

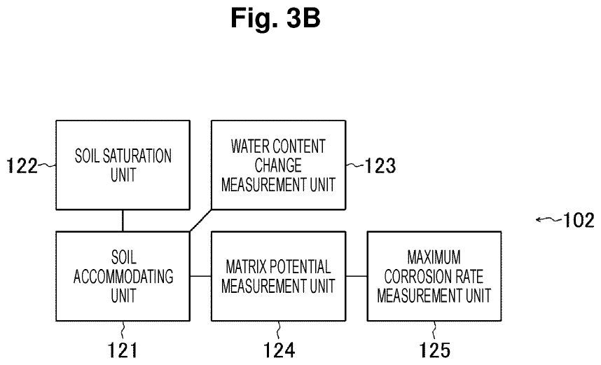

As illustrated in FIG. 3A, the soil conditioner 101 includes a soil accommodating unit 111, a drying unit 112, and a stirring unit 113.

The soil accommodating unit 111 stores a soil sample collected from the place where the metal member for which soil corrosion is to be estimated is buried. An amount of soil to be stored in the soil accommodating unit 111 may be any amount as long as the moisture characteristic measurement can be performed, and for example, it is preferable to prepare about 500 mL. A shape of the soil accommodating unit 111 is not limited as long as it is a storage container having a size capable of storing the above-described amount of soil. A material of the storage container constituting the soil accommodating unit 111 can be arbitrarily determined by a user.

In a case where the storage container constituting the soil accommodating unit 111 is metal, when the wet soil is stored, a corrosion reaction with the wet soil may occur depending on the type of metal, the storage container may be deteriorated, and a corrosion product may be mixed into the wet soil, and the original soil characteristic may be impaired. Therefore, it is preferable to avoid metal when selecting the material of the storage container constituting the soil accommodating unit 111.

In addition, when a method of applying heat is selected when the soil stored in the soil accommodating unit 111 is dried by the drying unit 112, it is preferable to avoid a material weak to heat. For example, a heat-resistant polymer resin or glass can be adopted as the material constituting the soil accommodating unit 111.

In a case where the soil stored in the soil accommodating unit 111 is wet soil, the soil particle mass may be formed by the water trapped in the soil particle gap. When the moisture characteristic is measured in the presence of the soil particle mass, the original moisture characteristic may not be evaluated because the particle size distribution is different. In order to remove the water that is the cause of the soil mass, the drying operation of the soil in the soil accommodating unit 111 is performed in the drying unit 112. As the drying method in the drying unit 112, for example, heat can be applied to increase a temperature of the soil accommodating unit 111 and dry the soil. Further, the inside of the soil accommodating unit 111 can be decompressed and vacuum-dried.

However, in the case of adopting the method of drying by applying heat, it is necessary to select a material that the soil accommodating unit 111 can endure up to a temperature set by the user. In addition, in a case where the soil in the soil accommodating unit 111 contains humus and exhibits a black color, there is a possibility that chemical components derived from organic substances are modified by heat and properties inherent in the black soil are lost. Therefore, the heating temperature as the pretreatment is preferably kept at an upper limit of 50° C. or lower.

In addition, when the decompression method is adopted, the storage container used for the soil accommodating unit 111 needs to be a material that can withstand decompression. Therefore, when the decompression method is used, the material of the storage container is preferably glass.

The drying operation by the drying unit 112 is ended when the soil water content in the soil accommodating unit 111 reaches 0%. For example, by installing a water content sensor in the soil accommodating unit 111, it is possible to measure the water content of the soil stored in the soil accommodating unit 111. The method for drying the soil in the drying unit 112 is not limited to the methods described above as long as the soil water content in the soil accommodating unit 111 can be set to 0%.

The stirring unit 113 performs stirring for unraveling the soil particle mass in the soil in the soil accommodating unit 111 in which the soil water content has reached 0% by the drying by the drying unit 112. The stirring of the soil in the stirring unit 113 is not limited as long as it is a method in which all the soil particle masses are eliminated. For example, stirring can be performed by the stirring unit 113 by stirring two rod-shaped objects in a circle. In addition, a mechanism similar to an automatic stirrer adopted in a food factory or the like can be adopted as the stirring unit 113.

As illustrated in FIG. 3B, the measurement instrument 102 includes a soil accommodating unit 121, a soil saturation unit 122, a water content change measurement unit 123, a matrix potential measurement unit 124, and a maximum corrosion rate measurement unit 125.

The measurement instrument 102 measures the moisture characteristic of the soil subjected to the pretreatment step by the soil conditioner 101. First, the pretreated soil is transferred from the soil accommodating unit 111 of the soil conditioner 101 to the soil accommodating unit 121. An accommodating container used for the soil accommodating unit 121 is not particularly limited in shape, material, and the like as long as the accommodating container has a capacity capable of storing all the pretreated soil in the soil accommodating unit 111 and is capable of measuring the moisture characteristic of the soil. In addition, in a case where an electrochemical measurement in the maximum corrosion rate measurement unit 125 is performed, it is necessary to bury an electrode (for example, a metal electrode) when the soil is transferred from the soil accommodating unit 111 to the soil accommodating unit 121.

The soil saturation unit 122 performs a treatment of removing a gas phase of the soil particle gap from the soil stored in the soil accommodating unit 121 and entirely filling the soil with a liquid phase, that is, saturating the soil. Examples of the saturation treatment include a water immersion deaeration method and a water absorption deaeration method. The water immersion deaeration method is a method in which the soil accommodating unit 121 is placed in a water immersion decompression container filled with water, and the inside of the water immersion decompression container is gradually decompressed by a vacuum pump or the like to be saturated. When the generation of air bubbles from the soil accommodating unit 121 is no longer confirmed, that is, after the gas phase in the soil is entirely filled with the liquid phase, a pressure in the container is gradually returned to an atmospheric pressure to complete the treatment.

In the water absorption deaeration method, the soil accommodating unit 121 is connected to a decompression water supply device, and decompression of the inside of the soil accommodating unit 121 and water supply from a water supply bottle to the soil accommodating unit 121 are alternately performed by a vacuum pump or the like. The treatment is completed by repeating the above-described decompression and water supply until no air bubbles come out of an aspirator bottle constituting the vacuum pump. There is no limitation as long as the method can saturate the pretreated soil in the soil accommodating unit 121.

The water content change measurement unit 123 measures a water content change of the soil subjected to the soil saturation treatment in the soil accommodating unit 121. The soil in the soil accommodating unit 121 has the maximum soil water content by the saturation treatment of the soil saturation unit 122. The water content sensor is inserted into the saturated soil, and a temporal change of the soil water content is measured. In the measurement of the temporal change of the soil water content, a filtration filter is installed on a bottom surface of the soil accommodating unit 121 in order to reproduce drainage of an actual environment. A pore size of the filtration filter is not limited, but if the pore size is too fine, a rate at which water escapes from the filter is slow, and a rate of drainage may be underestimated. Therefore, the pore size of the filter is preferably about 50 μm.

In the measurement by the water content change measurement unit 123, the soil accommodating unit 121 is held at a constant temperature and a constant humidity environment. As this environment, for example, the annual average temperature and the annual average humidity in the Kanto region can be adopted. The saturated soil is allowed to stand under the constant temperature and the constant humidity environment, the soil water content is gradually reduced by natural drainage, and the temporal change of the water content is measured by the water content sensor.

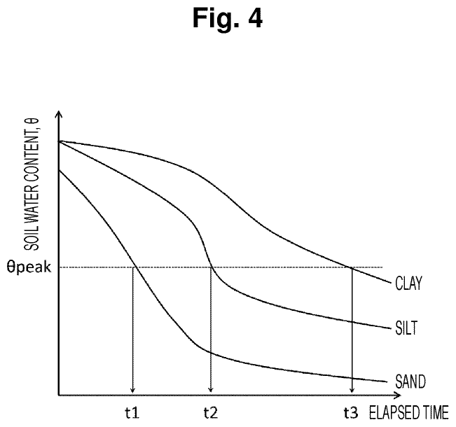

FIG. 4 illustrates an example of a graph representing the temporal change of the soil water content in the soil accommodating unit 121. FIG. 4 illustrates three types of sand, silt, and clay, and illustrates an example in which the sand, silt, and clay contain larger soil particles in that order, so that the water content decreases earlier in that order.

The matrix potential measurement unit 124 measures a relationship between a matrix potential representing how much the water in the soil is held by a strong force with respect to the soil in the soil accommodating unit 121 saturated by the treatment of the soil saturation unit 122 and the soil water content. When a maximum corrosion rate described later is measured, the metal electrode is buried in advance before the measurement is performed.

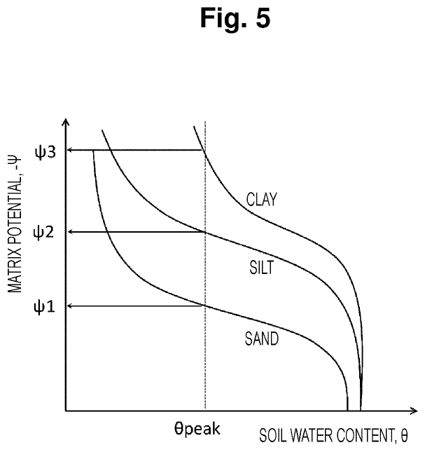

FIG. 5 illustrates an example of a soil moisture characteristic curve representing the relationship between the matrix potential and the soil water content. The unit of the matrix potential is Pa, which indicates that water is held with a strong force when a value is small, that is, when an absolute value is large. FIG. 5 illustrates the three types of sand, silt, and clay. When the matrix potential is a value close to zero, the soil is saturated, and mainly water is retained by the surface tension of the soil particle gap. As the soil water content decreases, the matrix potential becomes smaller, and water is strongly adsorbed to a small soil particle gap.

In FIG. 5, in the sand, water is mainly held by capillary force, and most of the water is drained at a high matrix potential. On the other hand, in the clay, water is retained by intermolecular force and force due to osmotic pressure in addition to capillary force, so that it can be seen that the water does not escape to a smaller matrix potential. For the measurement of the matrix potential and the creation of the moisture characteristic curve, for example, a moisture characteristic curve/unsaturated hydraulic conductivity coefficient measurement device HYPROP 2 and a matric potential measuring device WP4C manufactured by METER can be used. As long as the matrix potential can be measured and the moisture characteristic curve can be created, the device is not limited to the devices described above.

The maximum corrosion rate measurement unit 125 performs electrochemical measurement for calculating the maximum corrosion rate of the soil. The electrochemical measurement is performed by using the metal electrode buried in the soil in advance. Details of a method of calculating the maximum corrosion rate or the like will be described later. A relationship between the maximum corrosion rate obtained by the maximum corrosion rate measurement unit 125 and the matrix potential obtained by the matrix potential measurement unit 124 is derived. The electrochemical measurement is not limited as long as a charge transfer resistance R_{ct} can be measured, such as with an impedance method or a direct current polarization resistance method. A conversion coefficient K for calculating a corrosion current density from the measured charge transfer resistance value is calculated by comparing with a weight change after the test, and the maximum corrosion rate is derived from the following Expressions (1) and (2).

$$\begin{matrix}Equation 1 & \\ {i}_{corr} = K \cdot \frac{1}{{R}_{ct}} & ( 1 )\end{matrix}$$

$$r = \frac{M}{z ρ F} \cdot {i}_{corr} \times {10}^{- 6} \qquad ( 2 )$$

In the Expressions (1) and (2), i_{corr} represents a corrosion current density [μA/cm²], K represents a conversion coefficient, R_{ct} represents a charge transfer resistance [Ω·cm²], r represents a maximum corrosion rate [mm/year], z represents an ionic valence, ρ represents a density [g/cm²], F represents a Faraday constant [C], and M represents an atomic weight [mol].

As will be described later, a process in the maximum corrosion rate measurement unit 125 may not be performed again after the relationship between the maximum corrosion rate and the matrix potential is derived. If the relationship is known from the beginning, the process in the maximum corrosion rate measurement unit 125 does not need to be performed.

The calculator 103 estimates the soil corrosion of the buried steel material based on a result obtained by the measurement instrument 102.

Results measured by the water content change measurement unit 123 and the matrix potential measurement unit 124 in the measurement instrument 102 are stored in a memory of the calculator 103.

The calculator 103 estimates the soil corrosion based on each measurement result saved (stored) in the memory. The progress of the soil corrosion reaction is determined by a wetted area of a surface of the buried metal member and an oxygen partial pressure. The wetted area depends on a potential of the water trapped in the soil particle gap of the soil. In the oxygen partial pressure, after the soil particle gap is filled with water such as rain, the water permeates and diffuses deep into the ground as gravitational water, and oxygen diffuses from a surface layer to the ground to supply oxygen to the surface of the metal member.

The oxygen can be dissolved in water and reach the surface of the metal member as dissolved oxygen, but since a diffusion rate of the dissolved oxygen is 10⁴ times slower than the diffusion rate of gaseous oxygen, the oxygen required for the corrosion reaction is more likely to be supplied, as a distance of diffusion in the soil as a gas is longer. Drainage in the soil progresses, and the water (liquid phase) in the soil particle gap is replaced by air (gas phase), so that the distance in which gaseous oxygen can be diffused becomes long.

Since there is a saturated state immediately after the start of the test, corrosion proceeds by a mechanism similar to that of aqueous solution corrosion, and a water film exceeding a limiting diffusion layer is formed, and the diffusion rate of the dissolved oxygen is rate-determined, indicating a constant corrosion rate. When the soil water content decreases and the liquid phase in the soil is replaced by the gas phase, oxygen is efficiently supplied while the wetted area of the surface of the metal member is secured, so that the corrosion rate rapidly increases and reaches the maximum corrosion rate. A maximum value of the corrosion rate should be higher as the water film formed on the surface of the metal member is thinner, and it is necessary for the wetted area of the surface of the metal member to be secured. As time further elapses and the soil water content decreases, the water film becomes thin, but the wetted area of the surface of the metal member cannot be secured, and the corrosion rate decreases.

Next, an output of the estimation result in the calculator 103 will be described. First, since a constant corrosion rate is maintained after the start of the test, the value is arbitrarily determined. If it is desired to determine a more accurate initial corrosion rate, an electrical resistance value of the saturated soil is measured, and a larger initial corrosion rate can be set if the electrical resistance value is small, and a smaller initial corrosion rate can be set if the value is large. Subsequently, a time at which the corrosion rate rapidly increases is set. In a water content change curve shown in FIG. 4, it is assumed that the corrosion rate rapidly increases at a certain constant soil water content, and a time when a soil water content θpeak is reached is recorded. In the example of FIG. 4, the sand is recorded as time t1, the silt as time t2, and the clay as time t3.

θpeak can be arbitrarily determined by the user, but θpeak is preferably set within a range of 10 to 30%, since the corrosion rate increases in a situation where the soil is dried from a saturated state and the liquid phase and the gas phase are present in a well-balanced manner. Since the rapid increase in the corrosion rate is often observed when the soil water content is 20%, θpeak=20% can be set.

Subsequently, in the moisture characteristic curve illustrated in FIG. 5, the matrix potential value at θpeak is recorded. In FIG. 5, the sand is recorded as a matrix potential value ψ1, the silt is recorded as a matrix potential value ψ2, and the clay is recorded as a matrix potential value ψ3.

Subsequently, the maximum corrosion rate is determined. When soil having the same water content is compared, in the soil having a high matrix potential, water is held only in a region where the soil particle gap is locally narrowed, and similarly, the wetting of the surface of the metal member locally forms a thick water film. As a result, the maximum corrosion rate in the soil having the high matrix potential becomes smaller.

On the other hand, in the soil having a low matrix potential, a narrow region of the soil particle gap is uniformly formed over the entire soil, and water is strongly retained, so that a water film that is thin and in which the wetting of the surface of the metal member is uniform is formed. This increases the maximum corrosion rate in the soil with the low matrix potential.

In fact, when metal was buried in sand and clay for the same period of time, corrosion products were generated only locally in sand with a high matrix potential and metallic luster was mostly observed, whereas corrosion products were generated on the entire surface of metal in clay with a low matrix potential. In view of these facts, when θpeak is reached, the matrix potential measurement unit 124 performs electrochemical measurement using the metal electrode buried in advance, and calculates the maximum corrosion rate from the obtained charge transfer resistance R_{ct}.

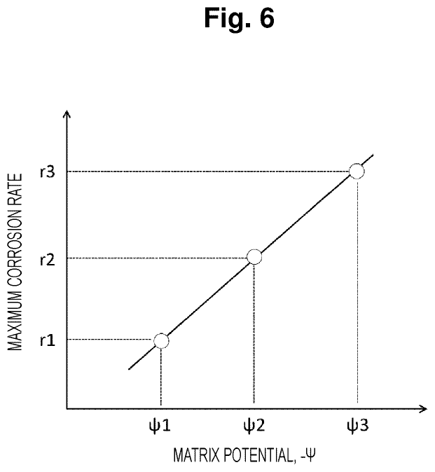

The calculated maximum corrosion rate and the matrix potential are plotted to derive the relationship between the matrix potential and the maximum corrosion rate illustrated in FIG. 6. The maximum corrosion rate calculation for plotting in FIG. 6 may be performed only for the first several samples, and if the relationship shown in FIG. 6 can be derived, the step of calculating the maximum corrosion rate can be omitted.

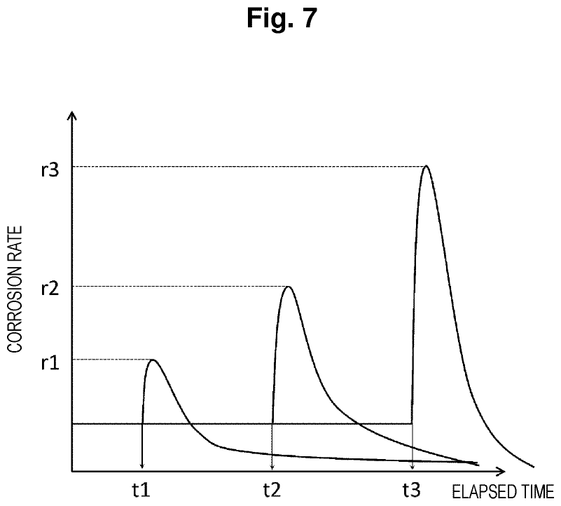

Based on the above results, a graph representing a temporal change of the corrosion rate shown in FIG. 7 is output. The time on the horizontal axis in FIG. 7 is linked with the time on the horizontal axis in FIG. 4, and the soil is saturated at a time of 0 hours. After the start of the test, the corrosion rate changes at a constant value, and at the time when the corrosion rate reaches θpeak, the corrosion rate rapidly increases to the maximum corrosion rate calculated from each matrix potential, and a peak of the corrosion rate appears. Thereafter, the corrosion rate decreases to a value of almost 0. A time at which the maximum corrosion rate is maintained can be determined with reference to the change in water content. For example, when θpeak=20% is set, it is assumed that the maximum corrosion rate is maintained until a 1% soil water content decreases, and the corrosion rate starts to decrease when the soil water content falls below 19%.

Next, estimation calculation of a corrosion amount is performed from information of the corrosion rate. Since the result illustrated in FIG. 7 shows the temporal change of the corrosion rate from when rain falls until the next rainfall, it is possible to calculate the corrosion amount developing in one rainfall by integrating the corrosion rate. Therefore, rainfall information of an area where used soil was buried is acquired, and the corrosion amounts progressing in one rainfall are added by the number of rainfalls to obtain a corrosion amount R progressing in one year. From R, by “D=RTⁿ. . . (3)”, a power law known as an empirical model for predicting corrosion development can be used.

In Expression (3), D represents a corrosion amount [mm], T represents aging [year] of the buried metal member, and n represents a corrosion evaluation value of a material. However, since the value of n is empirically said to be 0.4 to 0.6, an intermediate value of 0.5 can be adopted. It is possible to estimate the corrosion amount of the buried metal member, by introducing an aging value into T of Expression (3), the aging value describing how many years have elapsed since the buried metal member for which estimation of the corrosion amount is desired has been buried.

The operation of the soil conditioner 101, the measurement operation of the measurement instrument 102, and the determination operation of the calculator 103 can be controlled using a controller. For example, the controller determines the end time of the drying step and instructs the drying unit 112 of the soil conditioner 101 to end the drying treatment. Further, a stirring time of the dry soil in the stirring unit 113 is instructed.

For example, the controller determines that the saturation process by the soil saturation unit 122 is ended in the soil accommodating unit 121 of the measurement instrument 102, and instructs the water content change measurement unit 123 and the matrix potential measurement unit 124 to start various measurement processes.

For example, the controller determines data to be used for soil corrosion estimation among various measurement results saved in the memory of the calculator 103, and instructs transfer to the calculator 103.

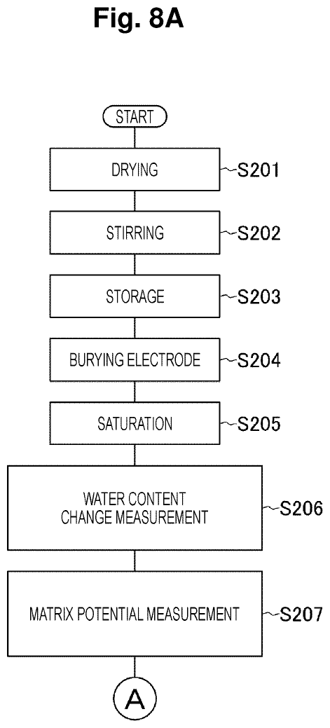

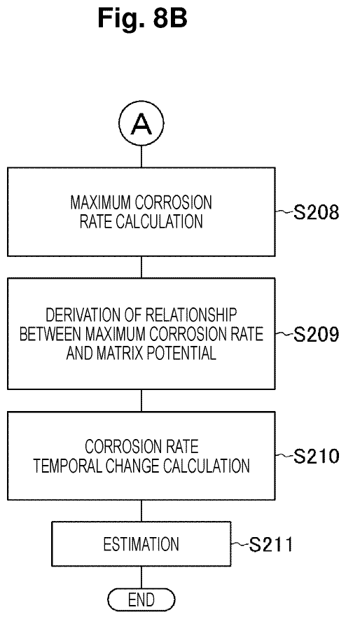

Next, an operation example (corrosion estimation method) of the corrosion estimation device according to the embodiment will be described with reference to FIGS. 8A and 8B. First, soil is stored in the soil accommodating unit 111 in the soil conditioner 101, and the soil is dried by the drying unit 112 (step S201). The stirring unit 113 stirs the soil in the soil accommodating unit 111 to remove the soil mass (step S202).

Next, the soil subjected to the above-described pretreatment is stored in the soil accommodating unit 121 of the measurement instrument 102 (step S203). In addition, an electrode is buried in the pretreated soil (step S204). Next, water is added to the pretreated soil in which the electrode is buried to perform the saturation treatment (step S205). For the saturated soil subjected to the saturation treatment, the water content change measurement unit 123 measures the temporal change of the soil water content from the saturated state to the dry state (step S206). Next, with respect to the saturated soil, the matrix potential measurement unit 124 measures the matrix potential and creates the moisture characteristic curve (step S207).

Next, when the specified soil water content is reached in the soil, electrochemical measurement is performed in the maximum corrosion rate measurement unit 125, and the maximum corrosion rate is calculated (step S208). Next, the relationship between the matrix potential obtained by the matrix potential measurement unit 124 and the maximum corrosion rate obtained by the maximum corrosion rate measurement unit 125 is plotted on a graph (step S209).

Next, the calculator 103 calculates a temporal change curve of the corrosion rate based on the measurement results of the water content change measurement unit 123, the matrix potential measurement unit 124, and the maximum corrosion rate measurement unit 125 (step S210). Next, the calculator 103 calculates the corrosion amount progressing in one year from the temporal change curve of the corrosion rate, and estimates soil corrosion from the power law expression (step S211). When the operation is ended as described above, the processing flow ends. When the relationship between the matrix potential and the maximum corrosion rate is derived, steps S204, S208, and S209 need not be performed again after being performed once, and the maximum corrosion rate can be calculated from the value of the matrix potential at the specified soil water content.

Note that the calculator 103 is, for example, a computer device including a central processing unit (CPU), a main storage device, an external storage device, a network connection device, and the like, and the CPU operates (executes a program) by a program developed in the main storage device, thereby realizing estimation of corrosion from the measured moisture characteristic. This program is a program for causing the computer to execute the third step of the corrosion estimation method. The network connection device can connect to a network and transmit the estimation result obtained in the third step to a device arranged at another point.

As described above, according to embodiments of the present invention, since the corrosion of the metal member is estimated from the moisture characteristic of the soil in which the metal member is buried, the corrosion of the metal member buried in the ground can be more accurately and easily estimated.

The present invention is not limited to the above-described embodiment, and it is apparent that various modifications and combinations can be implemented by those skilled in the art without departing from the technical spirit of the present invention.

### REFERENCE SIGNS LIST

101 Soil conditioner
102 Measurement instrument
103 Calculator

1-6. (canceled)

7. A corrosion estimation method comprising:
performing a pretreatment for measuring a moisture characteristic of soil;
measuring the moisture characteristic of the soil that has been subjected to the pretreatment; and
estimating corrosion of a metal member buried in the soil from the measured moisture characteristic.

8. The corrosion estimation method according to claim 7, wherein:
the moisture characteristic is a water content of the soil.

9. The corrosion estimation method according to claim 8, wherein:
the moisture characteristic is a matrix potential value at a specified soil water content.

10. The corrosion estimation method according to claim 7, wherein:
the moisture characteristic is a matrix potential value at a specified soil water content.

11. The corrosion estimation method according to claim 7, wherein performing the pretreatment comprises removing moisture from the soil.

12. The corrosion estimation method according to claim 11, wherein removing moisture from the soil comprises applying heat to the soil.

13. The corrosion estimation method according to claim 12, wherein applying heat to the soil comprises limiting a temperature of the soil to 50° C. or lower.

14. The corrosion estimation method according to claim 11, wherein removing moisture from the soil comprises decompressing and vacuum drying the soil.

15. A corrosion estimation device comprising:
a soil conditioner configured to perform a pretreatment for measuring the moisture characteristic of the soil;
a measurement instrument configured to measure the moisture characteristic of the soil that has been pretreated by the soil conditioner; and
a calculator configured to estimate corrosion of a metal member buried in the soil from the moisture characteristic measured by the measurement instrument.

16. The corrosion estimation device according to claim 15, wherein:
the measurement instrument is configured to measure a water content of the soil.

17. The corrosion estimation device according to claim 16, wherein:
the measurement instrument is configured to measure a matrix potential at a specified soil water content.

18. The corrosion estimation device according to claim 15, wherein:
the measurement instrument is configured to measure a matrix potential at a specified soil water content.

19. The corrosion estimation device according to claim 15, wherein the soil conditioner is configured to perform the pretreatment by removing moisture from the soil.

20. The corrosion estimation device according to claim 19, wherein the soil conditioner is configured to remove moisture from the soil by applying heat to the soil.

21. The corrosion estimation device according to claim 20, wherein the soil conditioner is configured to limit a temperature of the soil to 50° C. or lower during applying heat to the soil.

22. The corrosion estimation device according to claim 19, wherein the soil conditioner is configured to remove moisture from the soil by decompressing and vacuum drying the soil.
